(19) United States
(12) Patent Application Publication
(10) Pub. No.: US 2010/0151041 A1
(43) Pub. Date: Jun. 17, 2010

(54) Hypersaturated gas in liquid

(76) Inventor: C. Edward Eckert, New Kensington, PA (US)

Correspondence Address: ANDREW ALEXANDER, 821 Zubal Road, Apollo, PA, 15613-9222, US

(21) Appl. No.: 12/660,012

(22) Filed: Feb. 19, 2010

Related U.S. Application Data
(63) Continuation-in-part of application No. 11/857,556, filed on Sep. 19, 2007.
Application No. 11/857,556 is a continuation of application No. 10/197,787, filed on Jul. 18, 2002, now Pat. No. 7,288,574.
(60) Provisional application No. 60/306,309, filed on Jul. 18, 2001.

(51) Int. Cl. A61K 33/00; A61P 17/02; A61P 21/00

(52) U.S. Cl. 424/600

(57) ABSTRACT

Dispersing a gas in a liquid to provide a mixture of saturated, supersaturated or hypersaturated solution to provide a suspension of bubbles containing gas therein.

### CROSS REFERENCE TO RELATED APPLICATION

This application is a continuation-in-part of U.S. Ser. No. 11/857,556, filed Sep. 19, 2007, which is a continuation of U.S. Ser. No. 10/197,787, filed Jul. 18, 2002, which claims priority from U.S. Provisional Application No. 60/306,309, filed Jul. 18, 2001. The disclosures of the aforesaid applications are incorporated by reference in their entireties in the present application.

### FIELD OF THE INVENTION

The present invention relates to solutions of dissolved gas, and more specifically, to mixtures containing a solution of gas, e.g., O₂, and a dispersion of the gas in a liquid, e.g., water.

### BACKGROUND OF THE INVENTION

This invention relates to solutions having large quantities of dissolved gas, and more particularly to mixtures containing a solution having large quantities of gas and a dispersion of ultra-small micro-bubbles of the gas in suspension.

Oxygenated solutions are used in a variety of applications where elevated dissolved oxygen content is desired. In the medical community, it is generally known that the effect of oxygen on living tissue can be characterized by three regimes, namely, metabolic enhancement (growth accelerator), metabolic inhibition (growth arrest), and toxicity. In the former regime, oxygenated solutions can be used to accelerate the healing and regeneration rate of damaged tissue. Such wounds include cuts, lacerations, sores and burns on the face, arms, legs, torso and roof of the mouth. When wounds begin to heal, fibroblastic cells divide and spread throughout the wound area. The fibroblastic cells produce collagen, an important protein that facilitates healing. Supplying sufficient quantities of oxygen to the wound area significantly enhances fibroblast proliferation. In particular, the fibroblastic cells use amino acids hydroxylated with oxygen to synthesize collagen chains. In addition to treating wounds, oxygen is frequently used in topical applications for cleaning and revitalizing skin. In facial cleansing, dissolved oxygen assists in exfoliating dead skin particles from the skin surface. Dissolved oxygen has also been used to remove toxins, particulates and other occlusions in skin pores. In addition, oxygen has been used to revitalize skin cells by joining with protein molecules to nourish the cells and produce collagen.

The amount of oxygen initially dissolved into solution is largely dependent on the method used to dissolve the oxygen gas into solution. One common method for oxygenating water is the coarse bubble aeration process, which is a subset of aeration methods known categorically as air diffusion. Pressurized air or oxygen gas is introduced through a submerged pipe having small holes or orifices into a container of water. Gas pressure is sufficient to overcome the hydrostatic head pressure, and also sustains pressure losses during passage through the small gas orifices. As a result, bubble aeration occurs at relatively low pressures; this pressure being predominantly a function of tube immersion depth.

Since all interphase interfaces have a characteristic surface energy, the creation of interfacial (surface) area is an energetic process. As a gas passes through an orifice, for example, pressure energy is converted to kinetic energy, which consequently satisfies the energetic requirements of the system for the production of surface area. Area and velocity are inversely proportional; hence, as the orifice diameter decreases, the corresponding pressure drop and gas velocity increase, and more surface area is generated. Smaller bubbles result. This process has a limiting condition, however, in that the amount of heat (as irreversible work) that is produced is inversely proportional to the square of orifice diameter. It therefore becomes impractical and energetically inefficient to operate at exceptionally small orifice diameters. This process also has an absolute limit as a gas velocity of Mach one is approached within the pore. Because a pore lacks the convergent/divergent geometry required to achieve supersonic flow, increasing pressure beyond the critical pressure will not result in a further reduction of bubble size.

Since oxygen therefore is introduced into solution at relatively low pressures in the bubble aeration process, the oxygen bubbles are relatively large. As a result, the aggregate bubble surface area for a dispersion of bubbles produced by bubble aeration is relatively small. The limited surface area produced by bubble aeration limits the concentration of gas that can be dissolved into solution. Oxygen dissolution is a function of the interfacial contact area between gas bubbles and the surrounding medium, and bulk fluid transport (mixing) in the liquid phase. In particular, the rate of oxygen dissolution is directly proportional to the surface area of the bubbles. A dispersion of very small bubbles, e.g., bubbles having diameters in the order of 50 microns, will have a much larger total surface area than a dispersion of large bubbles occupying the same volume. Consequently, the rate of oxygen dissolution in bubbling aeration is limited by the size of the bubbles introduced into the solvent. Fluid mixing is also very limited in bubbling aeration because the only energy source available for agitation is the isothermal expansion energy of oxygen as it rises in the solution.

Oxygen dissolution in bubbling aeration is also limited by ambient pressure conditions above the solution. If the solution being aerated is exposed to atmospheric conditions, the dissolved oxygen concentration will be limited to the solubility limit of oxygen (at its partial pressure in air of 0.21 atm) under such conditions. The desirability of bubbling aeration is further hampered by equipment and energy requirements. Large blower units are used to force the gas bubbles into the carrying liquid. These blowers generate high-energy costs and often require special soundproof installations or other engineering costs.

Hydrogen peroxide is another popular source of oxygen used in topical applications and baths. Oxygen is easily derived from hydrogen peroxide, or H₂O₂, because an H₂O₂ molecule readily dissociates into water (H₂O) and an oxygen free-radical. The decomposition of H₂O₂ into water and oxygen free-radicals creates an enriched solution that facilitates dermal contact with oxygen. Hydrogen peroxide is distributed in various grades and concentrations that are specific to certain applications. Solutions of 3% and 6% hydrogen peroxide are commonly sold to consumers who use the solutions to disinfect cuts and clean skin areas. Solutions of 35% hydrogen peroxide are frequently added to spas and hot tubs to disinfect the water. Skin therapists use solutions of 35% hydrogen peroxide in oxygen baths to improve tissue regeneration and remove toxins from the dermis. Some topical creams contain stabilized forms of hydrogen peroxide intended to prevent free-radical formation and infections in skin.

Despite being a significant source of oxygen, hydrogen peroxide has been the subject of significant controversy when used in skin treatment applications. Some authorities claim that hydrogen peroxide is cytotoxic to human fibroblasts, due to the presence of free-radical oxygen. As a result, some medical professionals recommend additional dilution of hydrogen peroxide solutions to avoid their toxic effects on skin. Authorities also state that hydrogen peroxide reduces white blood cell activity. Still others have found that hydrogen peroxide slows wound healing by drying the wound, which destroys the exudate and leads to necrosis of skin tissue. Dry tissue also makes the wound area prone to bacterial growth and infection. As a result, hydrogen peroxide has drawn some questions as to its suitability for treating skin wounds and burns.

### SUMMARY OF THE INVENTION

It is an object of this invention to provide a novel system for dissolving gas in a liquid.

It is another object of this invention to provide a novel system for incorporating large quantities of gas in liquids.

It is yet another object of this invention to dissolve large quantities of molecular oxygen in water.

It is still another object of this invention to provide large quantities of molecular oxygen in water to provide a hypersaturated solution of metastable molecular oxygen water solution.

And still it is yet another object of this invention to provide an apparatus for dispersing large quantities of a gas in a liquid.

These and other objects will become apparent from a reading of the specification and claims and an inspection of the drawings appended hereto.

In accordance with these objects, there is provided a method of dispersing a gas in a liquid to provide a mixture of a saturated, supersaturated or hypersaturated solution of the gas in the liquid and to provide a suspension of bubbles containing the gas therein. The method comprises the steps of providing a liquid pumping means, a liquid for introducing to the liquid pumping means, and pumping the liquid to a pressure greater than 5 atms to provide a pressurized liquid. A gas is introduced to the pressurized liquid and dispersed therein to provide a solution having the said gas dissolved therein and having bubbles of the gas. The solution is then subjected to a shearing action to reduce the size of the bubbles to provide a highly hypersaturated liquid and bubbles of said gas having a diameter as low as 5 μm.

Based on the foregoing, an oxygenated mixture is provided having a dissolved molecular oxygen content well above the equilibrium limit at ambient conditions. The oxygenated mixture can supply a large amount of molecular oxygen in a medium that is not traumatic to skin tissue. Since the dissolution of oxygen into solution occurs under hyperbaric conditions, a large concentration of oxygen is dissolved into solution. The resulting solution can have a dissolved oxygen content as high as 200 mg/l. In one embodiment of the solution, an oxygen-enriched solution is accompanied by a dispersion of micro-bubbles held in suspension. In another embodiment, the oxygenated solution and micro-bubble dispersion are encapsulated in a Bingham Plastic.

A method for using the oxygenated solution in medical treatment is also provided. The method includes the step of filling a bath with oxygenated solution and a micro-bubble dispersion. Wounded areas or bruised areas of a patient, such as burned tissue, or bruised muscles or tissue, are submerged into the oxygenated solution and dispersion. The solution is allowed to enter tiny fissures or cavities in the wounded tissue. Some of the dissolved oxygen contacts the wounded tissue and aids in the regeneration of new tissue cells. As the solution is circulated in the tissue layers, the dissolved oxygen nucleates into fine micro-bubbles that attach to skin fragments. A volume change occurs upon nucleation of the oxygen bubbles. The dispersion of micro-bubbles and nucleating bubbles exfoliate damaged tissue layers and non-surgically remove dead, devitalized, contaminated and foreign matter from the tissue cells as the bubbles rise to the surface of the bath, further assisting in debridement and the regeneration of new tissue cells.

### BRIEF DESCRIPTION OF THE DRAWINGS

FIG. 1 is a cross sectional view of a two-phase mixture containing a gas enriched solution and micro-bubble dispersion in accordance with the present invention.

FIG. 2 is a frontal view of an alternate mixture in accordance with the present invention.

FIG. 3 is a flow chart showing steps of a method for generating and using a gas enriched solution and micro-bubble dispersion in accordance with the present invention.

FIG. 4 is a flow chart showing steps of an alternate method for generating and using a gas-enriched solution and micro-bubble dispersion in accordance with the present invention.

FIG. 5 is a flow diagram showing steps in the invention.

FIG. 6 is a more detailed flow diagram for introducing gas to the liquid.

FIG. 7 is a cross-sectional view showing a phase contactor for introducing gas to the liquid.

FIG. 8 is a dimensional view of the rotator in the phase contactor.

FIG. 9 is a view of a diffuser used in the phase contacts.

FIG. 10 is a cross-sectional view along the line A-A in FIG. 7.

FIG. 11 is a flow diagram of a second embodiment of the invention.

FIG. 12 is a schematic view of the second embodiment of the invention.

FIG. 13 is a hemoglobin saturation curve.

FIG. 14 shows saturated oxygen concentration in aqueous solution.

FIG. 15 is a schematic of the process.

### DETAILED DESCRIPTION OF PREFERRED EMBODIMENTS

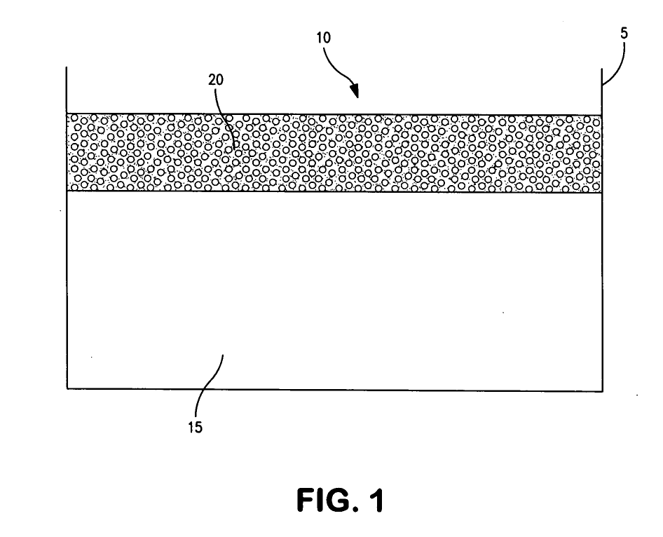

Referring to FIGS. 1-4 in general and FIG. 1 specifically, a two-phase mixture 10 containing a dissolved gas is illustrated. The mixture 10 contains a homogeneous solution 15 and a suspension or emulsion 20. The solution 15 contains a gas, such as molecular oxygen, dissolved in a solvent, such as water. The suspension 20 is formed by a dispersion of micro-bubbles containing a gas, such as molecular oxygen. For purposes of this description, the mixture 10 will be described as containing pure molecular oxygen gas in water. However, it is intended that the mixture may contain other solute gases and solvents, as will be discussed further below.

FIG. 1 shows the two-phase mixture in a static condition, where the mixture is stored in a vessel 5. The micro-bubble dispersion 20 consists primarily of molecular oxygen gas bubbles that have nucleated out of the solution 15. The micro-bubble suspension 20 has a lower density than the solution phase 15 and therefore forms a stratified layer on top of the solution. Although it is not clear from FIG. 1, the micro-bubble dispersion 20 typically has an occluded or cloudy appearance. This is caused by the scattering of visible light energy through the micro-bubble surfaces.

Referring again to FIG. 1, the homogeneous solution 15 will be described in further detail. The solubility limit of oxygen in water under equilibrium conditions with air (p_{O2}=0.21) at 77° F. is approximately 8.3 mg/l. When the two-phase mixture 10 is initially exposed to atmospheric conditions, the homogeneous solution 15 has a supersaturated or hypersaturated molecular oxygen content, i.e., above the solubility limit of oxygen in water under atmospheric conditions. Preferably, the homogeneous solution 15 has a dissolved oxygen concentration above 20 mg/l at 1 atm and 65° F. or higher. More preferably, the solution 15 has a dissolved oxygen concentration above 40 mg/l at 1 atm and 65° F. or higher. As a result, the oxygen concentration in the solution 15 is not stable when exposed to atmospheric conditions. Over time, exposure of the solution 15 to atmospheric conditions will cause some of the dissolved molecular oxygen to be lost through ebullition. More specifically, over time, dissolved oxygen molecules will gradually nucleate out of solution 15 into gas bubbles. Depending on pressure and temperature conditions, the concentration of dissolved oxygen will decrease down to the equilibrium concentration over a period of several minutes.

The supersaturated or hypersaturated molecular oxygen content in solution 15 is preserved by limiting agitation and preventing flow conditions in the solution that can facilitate ebullition of oxygen gases. The high dissolved oxygen content is also maintained by storing the solution 15 in a manner that limits or prevents desorption of the gas. For instance, the solution may be stored and distributed in sealed screw top containers constructed of glass or alternative materials impervious to oxygen diffusion at these high oxygen concentrations.

If oxygenated water is stored in capped bottles, made of an oxygen impervious material, elevated oxygen concentrations can be preserved for extended periods. In an experiment, seven glass bottles were filled with oxygenated water, processed as previously described, and immediately capped. A polargraphic probe was used to measure dissolved oxygen. The initial oxygen concentration was 64.2 mg/l, at a temperature of 17.6° C. Each bottle was uncapped for measurement of oxygen concentration at the intervals below:

| Initial | 6 hours | 1 day | 2 days | 3 days | 4 days |
|---|---|---|---|---|---|
| 64.2 mg/l | 65.7 mg/l | 63.5 mg/l | 67.5 mg/l | 58.5 mg/l | 55.4 mg/l |

It can be seen that over 86 percent of the original dissolved oxygen concentration was retained after 4 days. Such retention of oxygen in solution provides benefits in a number of applications. For example, a solution of oxygen dissolved in accordance with the above-described method may be used as an oxygen-enriched blood substitute.

As stated earlier, gas micro-bubbles that nucleate from solution, where the solution is a Newtonian fluid, such as water, rise to the surface and are released into the air above the solution. Gas bubbles rise in such fluids because a net body force exists that projects the bubbles upward. Since Newtonian fluids yield to these forces, the bubbles rise. These mechanics, which control bubble rise, are explained by Stokes Law, which will be examined later. In some applications, it is desirable to limit or substantially prevent bubbles from rising to the surface of the solution during storage and to maintain the micro-bubble dispersion indefinitely. In particular, it may be commercially desirable to market a product that contains visible oxygen bubbles that are held indefinitely in a suspension.

A supersaturated or hypersaturated solution of molecular oxygen in water is unstable at ambient pressure by definition. If, for example, the ambient temperature and pressure conditions establish an equilibrium oxygen concentration of 8 mg/l, and an oxygenated solution containing 40 mg/l is prepared at 5 atmospheres pressure, such a solution will have an oxygen concentration of 32 mg/l above the solubility limit. The oxygen-water system will attempt to reject oxygen by nucleating oxygen bubbles. Nucleation can be either a homogeneous or heterogeneous process, depending on changes in temperature, mechanical agitation, or the presence of suitable particles that can stimulate gas nucleation. Rapid pressure changes can provoke gas bubble nucleation, and in this invention, a reduction of pressure to ambient will typically result in the formation of micro-bubbles.

The micro-bubble dispersion 20 is characterized as having a very large surface area through which interfacial transport of oxygen occurs. Interfacial transport of molecular oxygen through a large surface area aids in resupplying oxygen to solution when dissolved oxygen is taken up during chemical reactions. As a result, a large surface area in the micro-bubble dispersion is desirable.

The mixture 10 preferably contains micro-bubbles having an average bubble diameter of about 2-9 μm or 10-100 microns. Micro-bubbles within this size range provide a significantly larger surface area than a cluster of large bubbles containing the same volume of gas. The magnitude of this difference can be visualized by performing calculations for several bubble diameters at a constant volume of gas. The following calculations show the surface areas present for a single bubble, a plurality of one-inch diameter bubbles and a plurality of 5 μm bubbles or 50-micron diameter bubbles, wherein each calculation is based on one cubic foot of gas. The value, r, is the radius of a single bubble, Vₒ is the volume of a single bubble, Aₒ is the surface area of a single bubble, and A is the aggregate surface area for the bubble formation:

a. Single bubble:

Vₒ=4/3πr³; r=(3Vₒ/4π)^{1/3}

Thus, when Vₒ=1.00 ft³, r=0.62 ft. Therefore, the diameter of a single bubble containing 1.00 ft³ of gas=1.24 ft.

The surface area of this single bubble (A_{b}) is:

A_{b}=4πr²=4π(0.62 ft)²=4.83 ft²

b: For one inch bubbles:

r=0.50 inches=0.042 ft.

The volume of a single bubble (V_{b}) is:

V_{b}=4/3πr³=4/3π(0.042 ft)³=3.1×10⁻⁴ ft³/bubble

A_{b}=4πr²=4π(0.042 ft)²=2.22×10⁻² ft²

The number of one inch bubbles (n_{b}) in a 1.00 ft³ volume of gas is:

n_{b}=Vₒ/V_{b}=1.00 ft³/3.1×10⁻⁴ ft³/bubble=3,224 bubbles

The surface area (Aₒ) of a 1.00 ft³ volume of gas comprised of one inch bubbles therefore is: Aₒ=ΣA_{b}=n_{b}A_{b}=3,224 (2.22×10⁻² ft)=71.43 ft²

c. For 50μ micro-bubbles:

r=25μ/3.05×10⁵ μ/ft=8.2×10⁻⁵ ft

V_{b}=4/3πr³=4/3π(8.2×10⁻⁵ ft)³=2.31×10⁻¹² ft³

A_{b}=4πr²=4π(8.2×10⁻⁵ ft)²=8.45×10⁻⁸ ft²

n_{b}=Vₒ/V_{b}=1.00 ft³/2.31×10¹² ft³=4.32×10¹¹ bubbles

Aₒ=n_{b}A_{b}=4.32×10¹¹(8.45×10⁻⁸ ft²)=36,504 ft²

d. For 5 μm micro-bubbles:

r=2.5μ/3.05×10⁵ μ/ft=8.2×10⁻⁶ ft

V_{b}=4/3πr³=4/3π(8.2×10⁻⁶ ft)³=2.31×10⁻¹⁵ ft³

A_{b}=4πr²=4π(8.2×10⁻⁶ ft)²=8.45×10⁻¹⁰ ft²

n_{b}=Vₒ/V_{b}=1.00 ft³/2.31×10⁻¹⁵ ft³=4.32×10¹⁴ bubbles

Aₒ=n_{b}A_{b}=4.32×10¹⁴(8.45×10⁻¹⁰ ft²)=365,800 ft²

Based on the foregoing calculations, the aggregate surface area for a dispersion of gas as bubbles increases by a factor of 10 as the radius of the bubbles decreases by a factor of 10. Referring to calculations (b) and (c), and within rounding error, a dispersion of 50-micron diameter bubbles containing one cubic foot of gas will have an aggregate surface area that is more than 500 times greater than a dispersion of one-inch bubbles containing the same volume of gas and a dispersion of 5 μm diameter bubbles containing one cubic foot of gas will have an aggregate surface area that is 10 times greater than the 50-micron diameter bubbles and 5,120 times greater than a dispersion of one-inch bubbles containing the same volume of gas.

The micro-bubble suspension 20 is unstable, as the micro-bubbles tend to rise to the surface of the mixture and pass into the atmosphere over time. This movement is generally driven by buoyancy (body) forces. The mechanics of micro-bubble separation in a liquid can be analytically described by Stokes' Law for small bubble sizes: V=2 gr²(δ_{g}δ_{w})/9η, where V is the terminal velocity of a bubble rising through the liquid, g is the acceleration of gravity, r is the radius of the bubble, δ_{g} is the density of the gas, δ_{w} is the density of the liquid, and η is the Newtonian viscosity of the liquid. Based on the formula, the terminal velocity of a rising bubble is proportional to the square of the radius of the bubble. In other words, the net upward force that causes the bubble to rise (i.e., the buoyancy force less all drag forces on the bubble) increases dramatically as the size of the bubble increases. For this reason, it is advantageous to minimize the size of the bubble so that the rate of bubble rise is minimized. Even with micro-bubbles that have diameters of 50 microns, however, the bubbles will nevertheless rise to the surface, releasing oxygen gas from the dispersion.

One novel aspect of this invention involves the substitution of a Newtonian solvent with a Bingham Plastic. Such a material requires a finite yield stress to initiate movement, and is described by the following equation: τ=+/−τ₂ηₚγ, where, τ=shear stress, τₒ=yield stress, ηₚ=plastic viscosity, and γ=strain rate. An important characteristic of a Bingham Plastic is that the yield stress, τₒ, must be exceeded before flow, or strain, γ, can occur. Applied stress levels that are below the yield stress threshold will not result in movement of the fluid. A Bingham Plastic can be considered to have infinite viscosity and behave as a solid at stress levels below the yield stress.

It can be seen from Stokes' Law, V=2 gr²(δ_{g}−δ_{w})/9η, that the limit of terminal velocity, V, is zero as the value for viscosity, η, approaches an infinite number. A Bingham Plastic will therefore result in bubble immobilization, provided that the magnitude of the buoyancy forces, 4/3Πr³(δ_{g}−δ_{w})g, exerts a stress level that falls below the yield stress for the Bingham Plastic. Bubble immobilization will provide stability of the micro-bubble suspension.

It has been discovered that the current invention can produce stable suspensions of micro-bubbles when a Bingham Plastic is used as the continuous, or solvent, phase. This is preferably accomplished by adding and mixing the ingredients to form a Bingham Plastic and an oxygenated liquid at elevated pressure, i.e., prior to the formation of micro-bubbles. A high-pressure mixer, that is downstream of the oxygenation process, can be used for this purpose. Since the components are mixed prior to the solution being reduced to ambient pressure, micro-bubbles will not substantially form. Once the solution is reduced in pressure, micro-bubbles will form; however, these bubbles are immobilized by the previously formed Bingham Plastic.

A variety of Bingham Plastics provide a suitable solvent phase, including but not limited to formulations using clay based thickening agents, such as Optigel-SH® manufactured by Sud-Chemie, Inc., and formulations using polymeric based thickening agents, such as Carbopol® polymers manufactured by B.F. Goodrich Company. Where oxygen micro-bubbles are used, Optigel-SH® is a preferred solvent, because it contains an oxidation resistant substance. It has been found that oxygen micro-bubbles, immobilized in a Bingham Plastic using a polymeric thickening agent, can react with the polymer and slowly release heat as a result of the reaction. The extended contact time provided by bubble immobilization allows this oxidation reaction to occur.

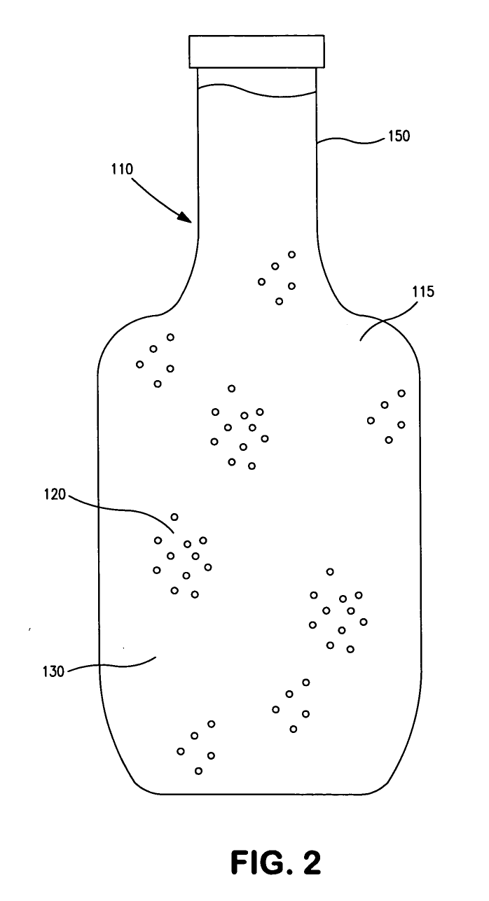

FIG. 2 illustrates a second embodiment of the present invention in which a Bingham Plastic 130 encapsulates a two-phase oxygenated mixture 110. The mixture 110 includes a homogeneous solution 115 of oxygen in water and a micro-bubble dispersion 120 contained in the Bingham Plastic 130. In FIG. 2, the mixture 110 is shown stored in a transparent bottle 150, which allows the oxygen gas micro-bubbles to be visible in the Bingham plastic 130 during storage. While the mixture 110 is shown stored in a bottle 150, the mixture is intended to be distributed in various types of containers, the choice of container being dependent on the type of product being marketed and the desired product configuration. The two-phase mixture 110 may be distributed with the Bingham Plastic 130 in a variety of products where there is a commercial interest in preserving the micro-bubble dispersion. For instance, the two-phase mixture and plastic may be marketed in shaving gels, hair gels, shampoos, ointments, lotions and other products.

The Bingham plastic 130 is characterized as having a finite yield stress. Fluid movement in a Bingham plastic 130 will not occur until the finite yield stress is exceeded. Once the yield stress has been exceeded, the stress may increase linearly with increasing shear rate. Buoyancy forces acting on the oxygen micro-bubbles 120 are insufficient to overcome the finite yield stress in the Bingham Plastic 130. Therefore, the Bingham Plastic 130 immobilizes the micro-bubbles 120 in the mixture for extended periods.

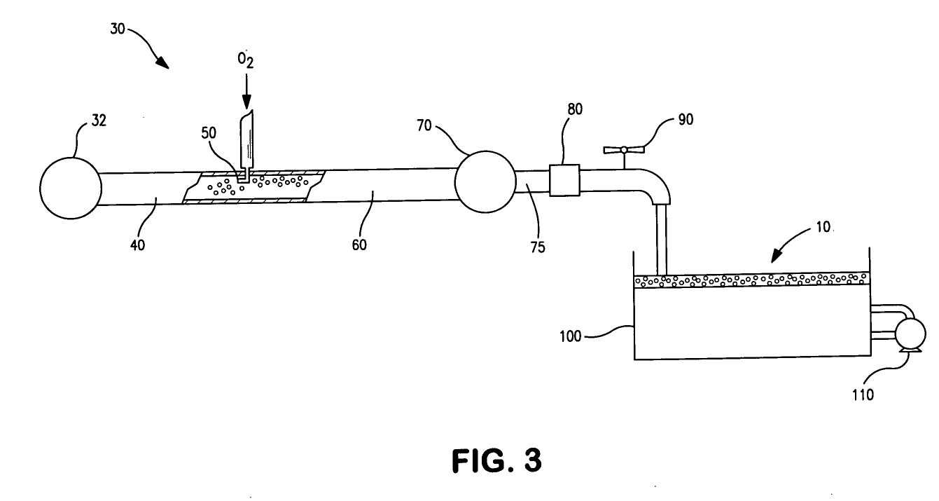

As stated earlier, the two-phase micro-bubble containing oxygenated mixture 10 can be used in any application in which molecular oxygen is beneficial, including the treatment of skin wounds, burns and reinvigorating fatigued muscles and bruised tissue. In one application, a skin wound may be submerged in the oxygenated mixture to non-surgically remove dead, devitalized, contaminated and foreign matter from tissue cells. Referring now to FIG. 3, a method for using the two-phase oxygenated mixture 10 in a bath 100 is illustrated. Water having a desired temperature is pumped through an oxygenation system 30. More specifically, the water is conveyed through a pre-charge pump 32 to pressurize the water. Preferably, the pressure of the stream is between 35 psig to 120 psig. In addition, the water preferably has a temperature no greater than 65° F., as warmer temperatures decrease the solubility of the gas in solution and may not be appropriate for the medical condition being treated. The water is discharged from the pre-charge pump 32 and conveyed to the oxygenation system 30 through an influent line 40, which is maintained at low pressure. Oxygen-containing gas is introduced into the influent line 40 from a supply of gas. In FIG. 3, oxygen gas is shown being injected into the liquid stream through a nozzle 50. The gas is injected substantially countercurrent to the flow direction in the influent line 40 at a high velocity. Countercurrent injection of the gas facilitates more complete mixing of the gas in solution, as a result of the instability of the jet plume. Injecting the gas at relatively high pressures further enhances mixing. Preferably, the gas is injected into the influent line 40 at a pressure between 150 psig and 450 psig.

In addition to oxygen, microbubbles can comprise at least one gas selected from the group consisting of air, molecular oxygen, hydrogen and nitrogen, atomic argon, helium, and neon. The bubbles can have an average diameter in the range of 1 to 40 μm.

Generation of micro-bubbles in the liquid stream requires a significant amount of energy. As a result, the gas must be introduced at a very high speed into the liquid. In the present method, the gas is preferably introduced at supersonic conditions at the exit of the nozzle 50. The nozzle 50 may be any type of nozzle that permits supersonic gas flow conditions, such as the nozzle disclosed in U.S. Pat. No. 5,463,176. The velocity of the gas at the exit of the nozzle 50 is preferably in the range of Mach 1 to Mach 5 and more preferably in the range of Mach 2 to Mach 4. It will be understood that lesser velocities, such as those below Mach 1, can be used but ordinarily will not provide as much mixing of gas into solution.

The introduction of gas at supersonic conditions into the low-pressure stream creates a two-phase oxygenated mixture 10. The mixture 10 is conveyed through a turbine based pump known as a co-compressor 70, which concurrently increases the pressure of both the gas and liquid in the stream and discharges the mixture into a high-pressure discharge line 75. The pressure of the gas and liquid are increased to allow large quantities of oxygen to efficiently dissolve in the liquid in a short period of time. The elevated pressure also substantially limits the remaining gas micro-bubbles from increasing in size. The amount of pressure in the discharge line 75 varies depending on the size of the system and desired discharge conditions. Preferably, the pressure of the mixture as it enters the discharge line 75 is between 150 and 800 psig. The high-pressure stream is conveyed to a discharge spigot 90 where it is discharged into a bath 100. Alternatively, depending on the pressure head in the high-pressure stream, the stream may be conveyed through a pressure reducer 80 prior to being conveyed to the discharge spigot 90, as shown in FIG. 3. The dissolved oxygen content in the mixture 10 at the point of discharge can be as high as 200 mg/l.

As the mixture 10 is discharged into the bath 100, the tank is allowed to fill with minimal agitation or stirring so as to substantially minimize the amount of nucleation and ebullition of gas bubbles. In this way, the high dissolved molecular oxygen concentration in the mixture 10 may be substantially preserved. Preferably, the bath is filled so that the dissolved oxygen concentration is kept above 20 mg/l at 1 atm and 65° F. Once the bath 100 is filled, the oxygenation system 30 and spigot 90 are turned off, and the patient or the patient's wounded areas are carefully placed in the bath. The solution is allowed to enter tiny fissures or cavities in the wounded tissue. Some of the dissolved oxygen contacts the wounded tissue and aids in the regeneration of new tissue cells. As the solution is circulated in the tissue layers, the dissolved oxygen nucleates into fine micro-bubbles that attach to skin fragments. These micro-bubbles exfoliate damaged tissue layers and carry them to the surface of the bath, assisting in debridement and regeneration of new tissue cells.

Although the elevated dissolved oxygen content in the bath 100 is not stable under atmospheric conditions, in the absence of bubble nucleation, the rate of oxygen liberation at the liquid/atmosphere interface is slow enough that the dissolved oxygen content in the bath can remain elevated for several hours. After this time, the dissolved oxygen content will decrease down to equilibrium conditions. Preferably, ambient pressure at the location of the bath is maintained between 0.9 atm and 1.1 atm.

Energy may be added to the bath solution after the bath is filled to stimulate the nucleation of micro-bubbles and accelerate the exfoliation process. For instance, heat energy may be added to promote homogeneous nucleation. Mechanical mixing or circulation of the bath solution using stirring bars, circulation pumps or other mechanical devices may also stimulate nucleation of micro-bubbles. In FIG. 3, a circulation pump 110 is shown which gently draws solution from the bath and recirculates solution into the bath. In some cases, heat dissipation from the submerged tissue may be sufficient to promote nucleation of micro-bubbles in the proximity of the tissue. Moreover, the addition of solid surfaces in the bath may be used to stimulate heterogeneous nucleation of micro-bubbles.

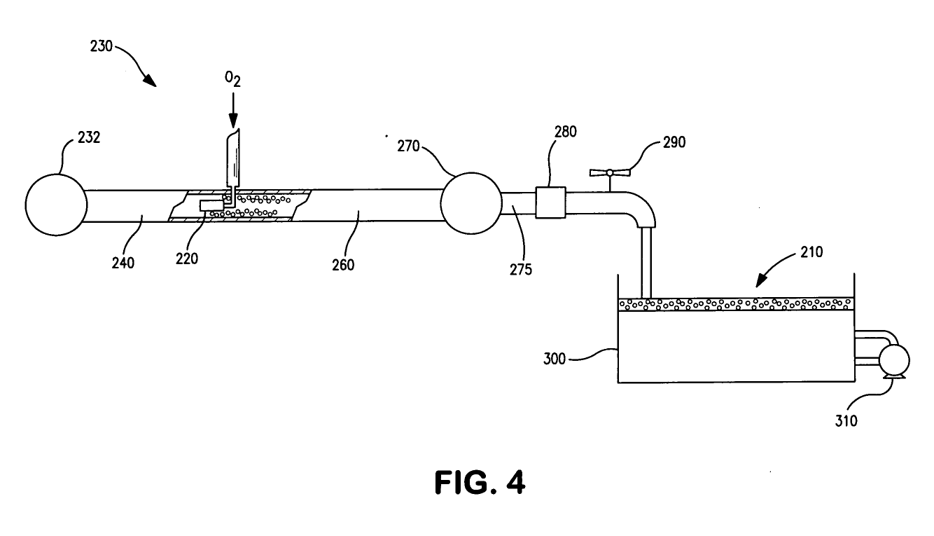

As an alternative to high velocity injection through nozzles, porous gas diffusion devices, such as sintered metal diffusers available from Mott Metallurgical, Inc., can be used to introduce gas into the liquid. Referring now to FIG. 4, an alternate method for making a two-phase oxygenated mixture 210 is illustrated. In general, components that are similar or identical to components in FIG. 3 are identified by the same reference number plus 200. Water having a desired temperature is pumped through an oxygenation system 230. The water is conveyed through a pre-charge pump 232 to pressurize the water. Preferably, the pressure of the stream is between 35 psig to 120 psig. In addition, the water preferably has a temperature no greater than 65° F., as warmer temperatures decrease the solubility of the gas in solution and may not be appropriate for the medical condition being treated. The water is discharged from the pre-charge pump 232 and conveyed to the oxygenation system 230 through an influent line 240, which is maintained at low pressure. Oxygen-containing gas is introduced into the influent line 240 through a porous diffusion device 220 connected to a supply of oxygen gas. The diffusion device 220 may have various geometries and be placed in a variety of ways in contact with the liquid. In FIG. 4, a cylindrical diffusion device 220 is shown disposed inside the influent line 240. Gas is delivered through the diffusion device 220 and into the pressurized liquid through a plurality of pores disposed through a the cylindrical face of the diffusion device. Preferably, the pores are no larger than 2 microns in diameter to facilitate the formation of small bubbles of the gas. The pressurized liquid flows past the diffusion device 220 in a direction transverse to the axis of the pores on the diffusion device 220 to create shear stresses along the outlets of the pores. As such, the shear stresses overcome attachment forces and surface tension that hold the micro-bubbles on the diffuser to detach and transport micro-bubbles as soon as they are formed on the diffuser face. In this way, the coalescence of large bubbles on the surface of the diffuser 220 is minimized.

The terms and expressions, which have been employed herein, are used as terms of description and not of limitation. There is no intention in use of such terms and expressions of excluding any equivalents of the features shown and described or portions thereof. It is recognized that various modifications of the embodiments described herein are possible within the scope and spirit of the invention. While the two-phase oxygenated mixture has been described primarily in terms of its use in skin products and topical treatment, the invention is intended for use in any application where a supply of oxygen is desired. For example, the oxygenated solution may be used to enhance tumor treatment or as an oxygen-enriched blood substitute. In the former case, oxygen may increase the chemo-sensitivity or radio-sensitivity of tumor cells, allowing a malignant condition to be more amenable to treatment. As a blood substitute, the oxygen-enriched solution can be administered intravenously in situations where whole blood products are not required. An example of such use is in response to blood loss due to hemorrhage, where fluid and oxygen are in critical need.

Reference herein to oxygen is meant to include molecular oxygen, but reference to molecular oxygen is meant to include only molecular oxygen or diatomic oxygen, or non-free radical.

A process has been developed to produce stable aqueous solutions of dissolved molecular gas, e.g., oxygen, with solute concentrations exceeding 1500 ppm or even 2000 ppm. Such solutions will be subsequently referred to as hyper-saturated aqueous solutions (HSAS). It can be shown through a Henry's Law based calculation that the resultant solute activities (10⁻³) approach the hyperbaric pressure equivalent of 80 atmospheres and higher. HSAS are metastable at ambient pressure under certain conditions if the energetic requirements for homogeneous nucleation of gas bubbles are not satisfied. Thus, aqueous solutions with an oxygen tension equivalent to saturation at 60-80 atmospheres and greater can be produced and maintained at ambient pressure for a reasonable time interval.

Numerous applications exploit high oxygen tension aqueous solutions. The medical applications include: (1) tissue regeneration/wound healing—As noted, it is generally known that the effect of oxygen on living tissue can be characterized by three regimes, namely, metabolic enhancement (growth accelerator), metabolic inhibition (growth arrest), and toxicity. In the former regime, oxygenated solutions can be used to accelerate the healing and regeneration rate of damaged tissue; (2) oxygen uptake (local and systemic)-trans-cutaneous oxygen transport is theoretically possible with sufficient oxygen tension. If such transport follows Fick's first law of diffusion, the diffusion flux is proportional to the concentration gradient, which is elevated in this case. Diffusion may be further enhanced by hydrophilic conditions. Trans-cutaneous transport could therefore constitute an alternative oxygen uptake route, and can be capable of supplementing respiration. On a local level, oxygen clearance in tissue could occur in ischemic situations since the local oxygen concentration is not dependent on circulation. Additionally, the introduction of oxygenated solutions to highly vascular areas, such as the peritoneum, could theoretically augment respiration as an oxygen intake route. Complete pulmonary off-load could be accomplished by an extra-corporeal device based on hydrophobic membranes; (e) burn debriedment; (4) peritoneal dialysis; (5) CO clearance; (6) blood substitutes; and (7) antiseptic.

Oxygen concentration decay in HSAS has been found to be approximately 12% per month under the correct storage conditions. The losses associated with oxygen diffusion through containment wall are believe to be the dominant decay mechanism.

Another important application for HSAS is its benefit for trans-cutaneous oxygen transport to proximal capillaries. The tranport problem is one of serial resistances, namely, the HSAS boundary layer, HSAS/skin interface, skin, skin/capillary interface, capillary wall, and boundary layer within the capillary.

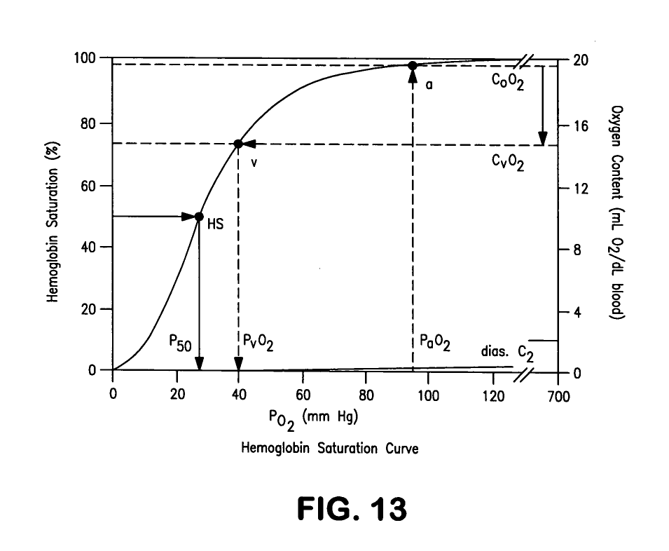

Hemoglobin may be taken as the dominant oxygen sink. Serum P_{O2} is essentially fixed by [O₂] in accordance with the hemoglobin saturation curve (see FIG. 13), and oxygen concentrations achieved in HSAS ([O₂]>100 ml O₂/dl water) compare very favorably to fully saturated oxyhemoglobin values. An oxygen diffusion flux driven by this concentration gradient can therefore be quite high, thus providing an efficient oxygen delivery means at the cellular level. Further, the oxygen capacity of HSAS exceeds saturated oxyhemoglobin by at least an order of magnitude.

Fick's first law relates an overall binary diffusion flux (J02) of oxygen in skin to the diffusivity tensor (Δ_{O2-T}) and the concentration gradient (Λω_{O2});

J_{O2}=−(ρ_{O2-T}.·.Λω_{O2})

If skin is considered as a homologous isotropic medium, then in situations involving a unidirectional flux in an isotropic media, the expression can be simplified to:

J_{O2X}=−ρD_{O2-T}(dω_{O2}/dx)

Two oxygen uptake mechanisms may be operative in traditional hyperbaric oxygen (HBO) treatment: inhalation route and cutaneous uptake. The latter mechanism involves the gas phase diffusion of oxygen to a stagnant water-laden surface, such as tissue or cell wall.

Since the water on these surfaces can be considered a thin film, gas/liquid interfacial area is considerable and transport distances across the layer are characteristically short.

Transport kinetics are consequentially favorable, provided that a high-rate oxygen sink reaction is present at the opposing interface. Hemoglobin oxidation will satisfy that requirement.

Aqueous phase oxygen transport within the stagnant interfacial layer is governed by ordinary diffusion with the diffusion flux proportional to the concentration gradient in accordance with Fick's first law. The concentration of oxygen in the aqueous solvent rapidly equilibrates with the ambient oxygen partial pressure, which is governed by Henry's law at dilute concentrations, and Sievert's law at higher oxygen concentrations. At three atmospheres of oxygen pressure, this oxygen equilibrium mole fraction is approximately 6.7×10⁻⁵, which equates to a dissolved molecular oxygen concentration of 120 ppm.

Higher oxygen concentrations require correspondingly higher gas pressure. Gas phase based HBO treatment has definite limitations on gas pressure defined by human tolerance. Further, the ability of a stagnant film to maintain an equilibrium relationship between oxygen pressure and dissolved oxygen concentration decreases with increasing oxygen pressure during a finite time interval.

This occurs because the oxygen dissolution reaction order shifts from zero order to first order, i.e., concentration dependent and mass transport limited. Aqueous phase oxygen transport within the stagnant interfacial layer is governed by ordinary diffusion with the diffusion flux proportional to the concentration gradient in accordance with Fick's first law. The concentration of oxygen in the aqueous solvent rapidly equilibrates with the ambient oxygen partial pressure, which is governed by Henry's law at dilute concentrations, and Sievert's law at higher oxygen concentrations.

Conventional gas phase HBO is limited to oxygen pressure in the range of 2.8-3 atmospheres. The concentration of dissolved oxygen in a HSAS is much higher than conventional HBO where system pressure can be increased to 200 atmospheres or more.

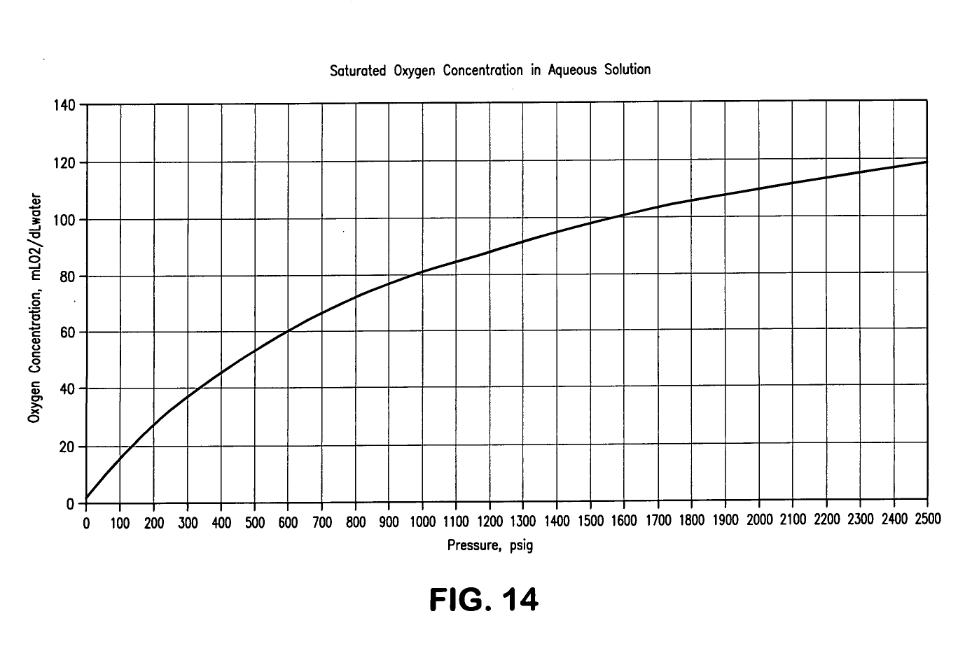

The concentration of dissolved oxygen in HSAS is a linear function of pressure at low concentrations and proportional to square root of pressure at higher oxygen concentrations. This concentration-pressure relationship for aqueous solutions of oxygen is illustrated in FIG. 14.

Thus, it is possible to create HSAS at elevated system pressures in accordance with this relationship. The solution free energy can also be derived from the relationship. Solutions prepared where the true equilibrium concentration of oxygen in water is established become supersaturated and metastable when pressure is reduced to ambient. It has been found that spontaneous homogeneous nucleation of oxygen bubbles does not occur with proper solution management and failure to supply activation energy (ΔG*) for nucleation.

It should be noted that O₂ is a molecule (O:O) with shared electrons and that O⁻ is an ion (free radical) with an unpaired electron. Water is formed from free radicals: H⁺+(OH)⁻=H₂O. Free radicals are usually reactive oxygen free radicals which can form from, for example, hydrogen peroxide: H₂O₂→H₂O+O⁻ or ozone: O₃=O₂+O⁻.

The process of achieving oxygen dissolution solution in HSAS at elevated pressure first involves the creation of a high surface area gas/liquid interphase interface. This is also an energetic process and is accomplished by creating a gas/liquid colloid resembling an emulsion. Molecular oxygen is not monomerized in the process.

As noted, common methods for oxygenating water include using gas diffusers at atmospheric and elevated pressures, packed beds and pressure tanks. Pressurized oxygen gas is introduced through a submerged pipe having small holes or orifices into a vessel of water. Gas pressure is sufficient to overcome the hydrostatic head pressure, and also sustains pressure losses during passage through the small gas orifices. As a result, bubble aeration occurs at relatively low pressures; this pressure being predominantly a function of tube immersion depth. The decrease in gas pressure across an orifice results in an increase in gas kinetic energy. This kinetic energy satisfies the energetic requirement to create surface area, albeit at a low level in this case. Since all interphase interfaces have a characteristic surface energy, the creation of interfacial (surface) area is an energetic process.

Area and velocity are inversely proportional; hence, as the orifice diameter decreases, the corresponding pressure drop and gas velocity increase, and more surface area is generated. Smaller bubbles result.

This process has a limiting condition, however, in that the amount of heat (as irreversible work) that is produced is inversely proportional to the square of orifice diameter. It therefore becomes impractical and energetically inefficient to operate at exceptionally small orifice diameters. This process also has an absolute limit as a gas velocity of Mach one is approached within the pore. Because a pore lacks the convergent/divergent geometry required to achieve supersonic flow, increasing pressure beyond the critical pressure will not result in a further reduction of bubble size. As noted earlier, a dispersion of very small bubbles, e.g., bubbles having diameters in the order of 50 microns, will have a much larger total surface area than a dispersion of large bubbles occupying the same volume.

One process uses a Mach 3 supersonic nozzle to dissipate energy at significantly higher levels than conventional orifice based oxygenation technologies. Oxygen issues from the nozzle countercurrent to water flow, resulting in the explosion of the jet plume into a quasi-emulsion. The gas liquid emulsion is subsequently pressurized to approximately 180 atmospheres for HSAS production.

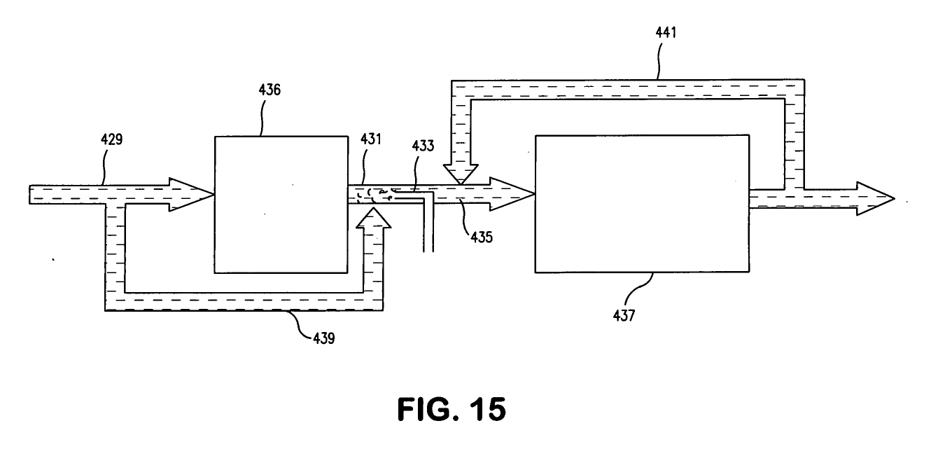

A schematic of the process is shown in FIG. 15 where water is introduced along line 429 to low pressure pump 436, which water is pumped to a low pressure, typically about 45 psig. The water is removed along line 431 where a gas, e.g., O₂, is introduced in counterflow direction through nozzle 433 to provide a mixture 435 of gas and water. The mixture is then introduced to a high pressure pump 437 where it can be pumped to pressures such as 300 psig or more to provide hypersaturated gas and liquid or oxygen and water which can be used directly or stored. Pump 436 is provided with a bypass 439 and pump 437 is provided with bypass 441.

Hyper-saturated aqueous solutions (HSAS) can have an important role in healing wounds. Acute and chronic wounds including ulcers, burns, sores, as well as crush injuries that occur on the face, torso, and extremities may benefit from treatment with HSAS. Also, such treatments aid in invigorating fatigued muscles or healing damaged tissue in the torso and extremities. Solutions of hyperbaric oxygen based on HSAS can provide significantly enhanced benefits as compared to traditional gas phase hyperbaric oxygen chambers. That is, traditional HBO operates at less than 3 atm oxygen pressure, resulting in a maximum solute oxygen concentration of 115 mg O₂/1 within cutaneous water. The oxygen potential (tension) of HSAS can exceed 2000 mg/l. HSAS will consequentially provide substantially higher oxygen diffusion flux. HSAS is inherently safer than conventional HBO since gas phase oxygen is not involved. Also, HSAS has the advantage that it is portable. Further, HSAS can produce volumetric expansion through the insitu heterogeneous nucleation of oxygen micro-bubbles. Such micro-bubbles, when formed within necrotic tissue can result in debriedment. In addition, treatment temperature can be easily manipulated. Advantageously, additional pharmaceuticals can be trans-cutaneously delivered concurrent with oxygen for possible synergistic benefit. Further, ultrasonic augmentation can be administered. Thus, HSAS has the benefit that it can therefore improve healing and recovery of fatigued muscles or damaged tissue through enhanced fibroblast proliferation, new vessel formation, and eradication of biofilms.

Therefore, HSAS can present a copious supply of oxygen directly and topically to ischemic wounds. It should be noted that hyperbaric oxygen appears to directly affect several aspects of skin healing. Traditional HBO has, in skin equivalent models been shown to stimulate the reconstruction of epidermis, stimulating proliferation, and early regeneration and remodeling of the basale membrane. Furthermore, the differentiation of keratinocytes and the remodeling of the basement membrane positively improve with conventional HBO therapy and therefore accelerate the remodeling process. Oxygen transport via water diffusion conduits can greatly improve oxygen levels to healing tissues not only through the intact stratum corneum but also wounded and burned skin. Keratin maintains hydration of the stratum corneum by minimizing evaporation losses. Augmented diffusion based oxygen transport can occur in HSAS treatment, not only in open wounds, but also skin with intact stratum corneum based on the ability to further hydrate through the adsorption of indigenous HSAS based water. Such water would also contain dramatically elevated oxygen levels.

Fibroblast supplying sufficient quantities of oxygen to the wound area significantly enhances fibroblast proliferation. As wounds begin to heal, fibroblastic cells divide and proliferate throughout the wound area. The fibroblastic cells produce collagen an important protein that facilitates healing. Specifically, the fibroblastic cells use amino acids hydroxylated with oxygen to synthesize collagen chains.

Vasculogenesis and angiogenesis in wounds have been shown to require oxygen. Data supports that oxygen impacts on angiogenesis in two ways: the first is to increase production of vascular endothelial growth factor (VEGF), and the second is through oxygen supporting the needs of growing vessels by facilitating the conversion of pro-collagen to collagen. Also, increased local oxygen tension at the cellular level would be expected to help maintain cellular metabolism through preservation or regeneration of cellular ATP levels, potentially increasing vasculogenesis angiogenesis, collagen synthesis and fibroblast function. This in turn would decrease the oxidative stress of persistent ischemia hastening proliferation of fibroblasts and maturation of keratinocytes in the re-epithelialization process. Further, pro-metabolic oxygen is expected to enhance angioblasts and circulating endothelial progenitor cells.

Biofilms also impact the chronicity of non-healing wounds. HSAS can play several roles in dealing with biofilms. Biofilms are a bacterial product that can be prevented or suppressed by the cytogenic effect of HBO directly on the bacteria. Secondly, the oxygen can react oxidatively with extra cellular matrix of the biofilm, which can structurally compromise its integrity. Thirdly, as noted earlier, the HSAS solution can assist in debriedment of the biofilm through sub-film nucleation of oxygen micro-bubbles, resulting in dramatic volumetric expansion, and promoting removal. HSAS can provide a simple method of removal in chronic wounds or burns.

Furthermore, HSAS can be efficacious in salvaging ischemic limbs using ingenious delivery methods trancutaneously and intravascular. These methods could save many ischemic limbs in conjunction with other available technologies.

HSAS has no requirements for special facilities; any increased safety risks, decreased cost, and potentially increased patient compliance. HSAS could play a synergistic role with other modalities to treat acute and chronic wounds. HSAS could hasten and improve healing of chronic wounds by improving the efficiency of all phases of healing including but not limited to the inflammatory, proliferative, remodeling and epithelialization phases. Improving on low mitotic activity, fibroblast proliferation and migration, and migration of keratinocytes among other benefits. Bacterial colonization of wounds could be decreased along with less biofilm presence. Tissue engineered dressing and skin grafts could then be applied to healthier tissue. Tissue grafts alone could then be treated with HSAS resulting in accelerated growth and healing.

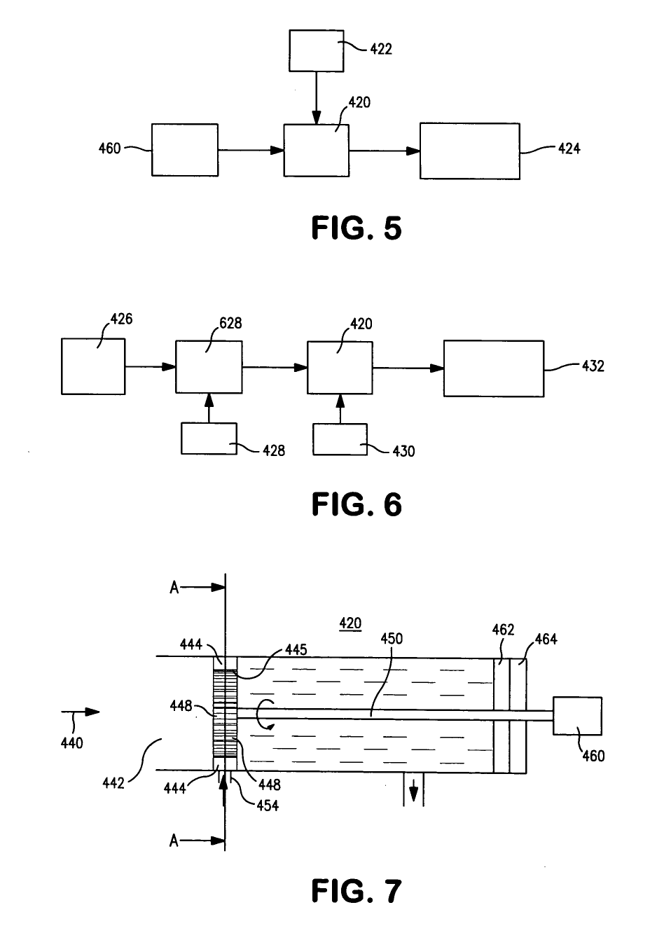

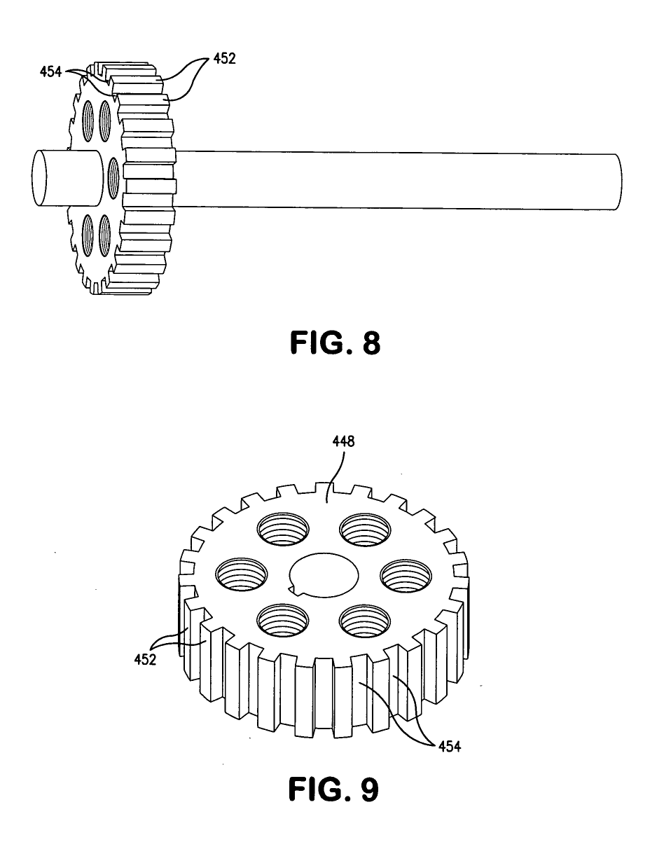

Referring now to FIG. 5, there is provided a general schematic or flow chart showing steps for producing HSAS in accordance with the invention. That is, in one aspect of the invention, high pressure liquid 400 is introduced to a phase contactor 420 where a gas from gas source 422 is dissolved in the liquid to provide very high levels of gas supersaturated in liquid 424.

FIG. 6 illustrates an embodiment of the invention to provide very highly saturated oxygen water solutions, for example, referred to herein as hypersaturated solutions (HSAS). That is, motor 426 turns pump 428 which pumps water from water source 427 to levels in the range of 5 to 320 or 20-80 atms. The high pressure water is introduced to phase contactor 420 where the water becomes hypersaturated with molecular oxygen from oxygen source 430 before being discharged to provide a solution 432 hypersaturated with molecular oxygen.

As noted earlier, introducing molecular oxygen to water through a porous defuser results in relatively large bubbles with low surface area. Increasing the pressure of the gas to provide an increased flow through the pores results in smaller bubbles. If a nozzle having convergent/divergent geometry is used, increasing gas flow rate is achieved and even smaller bubbles are obtained with resulting increased bubble surface area along with greater dissolution. Greater surface area of bubbles results in higher levels of dissolution of oxygen in water.

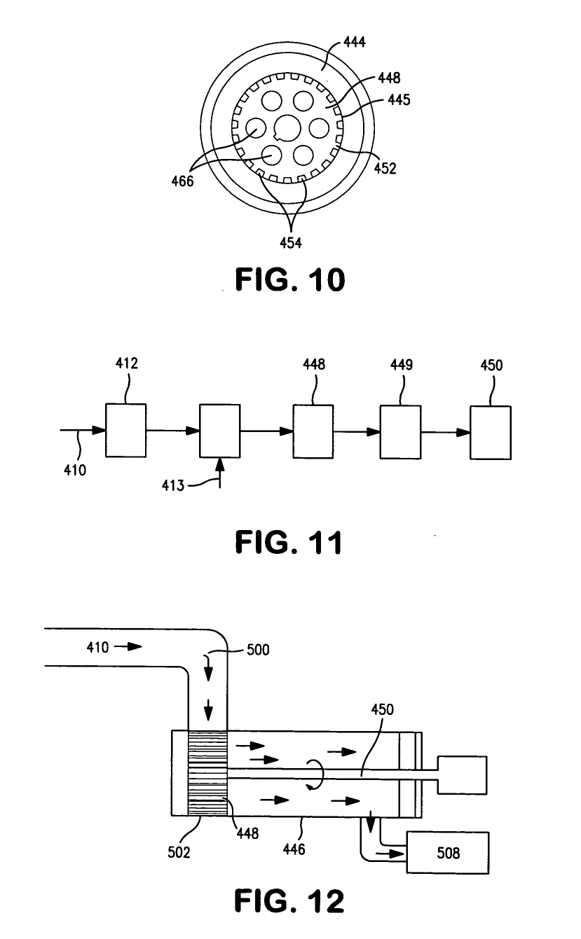

Referring now to FIG. 7, there is shown a phase contactor 420 capable of producing bubbles in the range of 5 to 250 μm diameter. In FIG. 7, high pressure water 440 is introduced to phase contactor 420 at end 442 and gas is introduced through ring diffuser 444. The gas is mixed with and dissolved in the water to provide a mixture of solution having gas dissolved therein and bubbles of gas. As shown in FIG. 7, a rotator 448, which is turned by shaft 450, is mounted to turn within inside surface 445 of diffuser 444. Rotator 448 is mounted within diffuser 444 to provide preferably a zero clearance fit, and yet the rotator should be permitted to rotate relatively freely. Rotator 448 (FIG. 10) is provided with teeth 452 which extend across width 450 of the diffuser. The teeth are separated by grooves 454. Diffuser 444 is comprised of a porous material to provide pores in a size range of 0.1 to 50 microns (broad size range, narrow size range). Gas is introduced through valve 454 and flows circumferentially through diffuser 444 and then radially inwardly to form gas bubbles in the liquid, particularly in grooves 454. As the rotator spins, it provides a shearing action with the result that bubbles forming are divided into much smaller bubbles, thereby increasing the surface area of the bubbles and greatly increasing dissolution of the gas in liquid.

Diffuser ring 444 may be formed from any suitable material that permits the flow of gas therethrough. Suitable materials are materials comprised of porous stainless steel, copper and alloys, nickel alloys, ceramics (Si₃N₄), porous carbon and titanium.

Rotator 448 may be formed from a metal or a plastic material. Preferably, the rotator is formed from stainless steel or Teflon®, titanium, nickel alloys, or ceramic may be used. During operation, the rotator would typically spin at a speed in the range of 75 to 5500 rpm. As will be seen in FIG. 7, shaft 450 is driven by electric motor 460. Shaft 450 is mounted on bearing 462 and sealed by end 464. As shown in FIG. 10, rotator 448 is provided with channels 466 which permits additional flow of liquid through the rotator.

Referring now to FIG. 11, there is shown an alternate method of obtaining hypersaturated gas-liquid combination. In this embodiment, liquid or water 410 is introduced to a low pressure pump 412. Low pressure pump 412 pumps liquid at low pressure and the gas 413 to be dispersed in the liquid is introduced to the low pressure water to provide a dispersion of gas and liquid. This, of course, will result in relatively large bubbles and low bubble surface area and the attendant low dissolution of gas in liquid. The dispersion of gas and liquid is then introduced to phase contactor 448, as shown in FIG. 11. Phase contactor 448 is turned at a speed up to 10,000 rpm and bubbles in the dispersion are finally divided to provide ultra-small diameter bubbles, e.g., 5 to 100 μm having a large surface area and high levels of dissolution of gas in liquid. The liquid having high levels of gas dispersed therein is subject to high pressures of 449, e.g., 5 to 320 atm, to dissolve most of the gas in the liquid which is sent to storage 450 or used as required. It should be understood that the rotator shown in FIG. 12 should have zero clearance to be effective. In this embodiment, the dispersion of the gas-liquid is preferably introduced perpendicular to the circumference of the rotator, as shown in FIG. 12. That is, the high pressure dispersion 500 should be introduced to the circumference 502 of rotator 448.

Slots 454 on rotator 448 are shown substantially parallel to the axis of the rotor 448. However, the slots may be provided at an angle to facilitate division of the gas bubbles in the solution to provide a supersaturated solution having very high levels of gas dissolved in the liquid.

With respect to FIG. 12, it should be noted that after the high pressure dispersion of gas-liquid, it is subject to treatment in the phase contactor, the hypersaturated solution is removed continuously at exit 506 to storage or use facility 508.

This process can produce stable aqueous solution of dissolved gas in solutions at very high concentrations, e.g., dissolved molecular oxygen with solute concentrations exceeding 1500 ppm, for example, 150 to 2000 mg/l. The resultant solute activities (10⁻³) can approach the hyperbaric pressure equivalent of 80 atms and higher. Such solutions are metastable at ambient under certain conditions because of the energy requirements for homogeneous nucleation of gas bubbles. Accordingly, aqueous solutions with an oxygen tension equivalent to saturation at 60 to 80 atmospheres can be produced and maintained at ambient pressure for reasonable time intervals.

As noted, the high oxygen aqueous solutions are useful for tissue regeneration, wound healing and invigorating fatigued muscles or restoring bruised tissue. Oxygen application to living tissue results in metabolic enhancement (growth accelerator) metabolic inhibition (growth arrest) and toxicity. Thus, highly oxygenated solution can be used to accelerate healing and regeneration rate of damaged tissue and fatigued muscles.

Oxygen uptake can be facilitated with sufficient oxygen tension and may be enhanced by hydrophobic conditions and high concentration gradients. Trans-cutaneous transport may provide an alternative oxygen uptake route and may supplement respiration. Also, complete pulmonary offload can be made possible by an extra corporeal device based on hydrophobic membranes.

A proposed uptake mechanism involves gas phase diffusion of the gas, e.g., oxygen, to a water laden surface such as tissue or cell wall.

The invention is further intended to encompass a wide range of solutes and solvents other than oxygen and water. For instance, injecting nitrogen gas into a solvent can form a two-phase mixture in accord with the present invention. When using the solution for skin debridement, a variety of gases may be dissolved into solution for safely debriding the tissue. A bath solution may be prepared using one or more gases, including, but not limited to air, carbon dioxide or a number of inert gases. Gas may be dissolved into or even reacted with a number of different solvents, such as propylene glycol or perflubrons to form a two-phase mixture. Accordingly, the present invention is not limited to the specific embodiments discussed above, but rather incorporates variations that fall within the scope of the following claims.

Reference to a range herein is meant to include all the numbers in the range, as if specifically set forth. For example, the range of 5-200 would include numbers 6, 7, 8 . . . 198, 199.

Having described the presently preferred embodiments, it is to be understood that the invention may be otherwise embodied within the scope of the appended claims.

What is claimed is:

1. An improved oxygen and water solution comprising dissolved molecular oxygen is in the range of 75 to 2000 mg/l, the remainder water.

2. The solution in accordance with claim 1 wherein the dissolved molecular oxygen is in the range of 150 to 1600 mg/l molecular oxygen.

3. The solution in accordance with claim 1 wherein the solution is obtained by subjecting small bubbles of molecular oxygen in water to a pressure of 5 to 320 atms of pressure.

4. The solution in accordance with claim 3 wherein the solution contains bubbles having an average diameter in the range of 5 to 250 μm.

5. An improved molecular oxygen and water solution containing 75 to 1600 mg/l molecular oxygen, the remainder water, the solution stable for a period of time at ambient temperature and pressure.

6. The solution in accordance with claim 5 wherein dissolved molecular oxygen is in the range of 150 to 1600 mg/l molecular oxygen.

7. The solution in accordance with claim 5 wherein the solution is obtained by subjecting small bubbles of molecular oxygen in water to a pressure of 5 to 320 atms of pressure.

8. The solution in accordance with claim 7 wherein the solution contains bubbles having an average diameter in the range of 5 to 250 μm.

9. A combination of molecular oxygen and water, the combination comprising 75 to 2000 mg/l dissolved molecular oxygen, 2 to 40 vol. % of suspended molecular oxygen bubbles having diameter of 5 to 250 μm, the remainder comprising water, minor concentrations of other elements and impurities.

10. The solution in accordance with claim 9 wherein the dissolved molecular oxygen component is within the concentration range of 75 to 650 mg/l, and the suspended molecular oxygen bubble content is 5 to 15 vol. % having a diameter less than 100 μm.

11. The solution in accordance with claim 9 wherein the solution is obtained by subjecting small bubbles of molecular oxygen in water to a pressure of 5 to 320 atms of pressure.

12. The solution in accordance with claim 11 wherein the bubbles have an average diameter of about 150 μm or less.

13. A multiphase mixture consisting essentially of:
(a) a liquid comprising water; and
(b) a plurality of microbubbles comprising at least one gas selected from the group consisting of air, molecular oxygen, hydrogen and nitrogen; and atomic argon, helium, or neon, the bubbles having an average diameter in the range of 1 to 40 μm.

14. The mixture in accordance with claim 13 wherein the mixture is saturated with molecular oxygen.

15. The mixture in accordance with claim 13 wherein the mixture is supersaturated with molecular oxygen.

16. The mixture in accordance with claim 13 wherein the mixture is hypersaturated with molecular oxygen.

17. The mixture in accordance with claim 13 wherein said mixture is stable at room temperature and pressure.

18. The mixture in accordance with claim 13 wherein said mixture will nucleate to form bubbles of gas.

19. A method of preparing a bath using a mixture of molecular oxygen and water by incorporating the molecular oxygen in the water, said method comprising:
(a) providing a body of water;
(b) introducing molecular oxygen to said water to provide a water solution containing bubbles of molecular oxygen;
(c) pressurizing the liquid and the bubbles to a pressure in the range of 5 to 320 atms to substantially cause the bubbles to dissolve into the water providing a dissolved molecular oxygen water solution containing molecular oxygen in the range of 75 to 2000 mg/l; and
(d) providing said dissolved molecular oxygen water solution in a container and forming a bath containing said dissolved molecular oxygen water solution.

20. A mixture of gas and water comprising:
(a) a water solution having molecular oxygen dissolved in water, the solution containing 75 to 2000 mg/l dissolved molecular oxygen; and
(b) a gas emulsion containing microbubbles of molecular oxygen, the bubbles having an average size in the range of 5 to 200 μm.

21. The mixture in accordance with claim 20 wherein said microbubbles exert a buoyant force.

22. The mixture in accordance with claim 20 wherein said microbubbles are encapsulated in a plastic, said plastic encapsulated bubbles having a yield stress greater than the buoyancy force exerted by said microbubbles.

23. The mixture in accordance with claim 22 wherein said plastic is a Bingham plastic.

24. The mixture in accordance with claim 22 wherein said plastic is a Bingham plastic which comprises one or more polymer components.

25. The mixture in accordance with claim 22 wherein said plastic is a Bingham plastic which comprises one or more clay components.

26. A method of treating a wound, comprising the steps of:
(a) dissolving a molecular oxygen into a water-containing liquid to form a solution under an elevated pressure condition to hypersaturate the dissolved molecular oxygen into the solution with respect to an ambient pressure and an ambient temperature, said elevated pressure being at least 10 atms, said solution containing 75 to 2000 mg/l molecular oxygen;
(b) transferring the solution to a container subjected to the ambient pressure and the ambient temperature;
(c) submerging tissue cells into the solution in the container;
(d) adding energy from an energy source to the solution to invoke nucleation of molecular oxygen containing microbubbles and liberation of the molecular oxygen from the solution in proximity to the tissue cells; and
(e) maintaining the tissue cells in the solution to non-surgically remove dead, devitalized, contaminated and foreign matter from the tissue cells by action of the microbubbles.

27. The method of claim 26 wherein the energy added to the solution comprises heat energy supplied to the solution.

28. The method of claim 27 wherein the heat energy supplied to the solution comprises heat dissipating from the tissue cells.

29. The method of claim 26 wherein the energy source for adding energy to the solution is mechanical circulation of the solution.

30. The method of claim 26 wherein the molecular oxygen is air or pure molecular oxygen.

31. The method of claim 26 wherein the step of maintaining the tissue cells in the solution further comprises enhancing proliferation of fibroblastic cells in the tissue cells through exposure of the cells to the molecular oxygen.

32. The method of claim 26 wherein the step of transferring the solution to a container comprises gradually reducing the pressure of the solution to minimize turbulent conditions and maintain the concentration of dissolved oxygen in solution above 100 mg/l during the transfer.

33. The method of claim 26 further comprising the step of maintaining the ambient pressure between 0.9 atm and 1.1 atm, and the ambient temperature between 65° F. and 72° F.

34. The method of claim 33 wherein the step of dissolving the molecular oxygen into the liquid comprises pumping the liquid through a conduit and injecting the gas into the pumped liquid at supersonic speeds.

35. The method of claim 26 wherein the energy source for adding energy to the solution originates from surface forces.

36. A method of treating a wound comprising the steps of:
(a) dissolving a molecular oxygen into water to provide a hypersaturated solution containing dissolved molecular oxygen substantially resistant to homogenous nucleation of molecular oxygen under static conditions in an ambient pressure and an ambient temperature environment;
(b) transferring the solution to an environment having the ambient pressure and the ambient temperature while adding a minimal amount of energy to the solution so as to maintain the concentration of dissolved gas in the solution as it is transferred to the environment;
(c) immersing tissue cells into the hypersaturated solution;
(d) adding energy from an energy source to the solution to induce nucleation of molecular oxygen microbubbles and liberation of the molecular oxygen from the solution in proximity to the tissue cells; and
(e) maintaining the tissue cells in the solution to non-surgically remove dead, devitalized, contaminated and foreign matter from the tissue cells by action of the microbubbles.

37. The method of claim 36 wherein the energy added to the solution comprises heat energy supplied to the solution.

38. The method of claim 36 wherein the heat energy added to the solution comprises heat dissipating from the tissue cells.

39. The method of claim 36 wherein the energy source for adding energy to the solution is mechanical circulation of the solution.

40. The method of claim 36 wherein the energy source for adding energy to the solution originates from surface forces.

41. The method of claim 36 wherein a solid surface is submerged in the solution to stimulate heterogeneous nucleation of the molecular oxygen containing bubbles.

42. The method of claim 36 wherein the step of maintaining the tissue cells in the solution further comprises enhancing proliferation of fibroblastic cells in the tissue cells through exposure of the cells to the dissolved molecular oxygen.

43. The method of claim 36 wherein the concentration of dissolved molecular oxygen in solution as it is transferred into the container is above 100 mg/l.

44. The method of claim 36 wherein, in the dissolving step, the solution of dissolved molecular oxygen is hypersaturated and has a dissolved molecular oxygen concentration above 100 mg/l.

45. The method of claim 42 comprising the step of maintaining the ambient pressure between 0.9 atm and 1.1 atm, and the ambient temperature between 65° F. and 72° F.

46. A method of revitalizing fatigued muscles or damaged tissue, comprising the steps of:
(a) dissolving a molecular oxygen into a water-containing liquid to form a solution under an elevated pressure condition to hypersaturate the dissolved molecular oxygen into the solution with respect to an ambient pressure and an ambient temperature, said elevated pressure being at least 10 atms, said solution containing 75 to 2000 mg/l molecular oxygen;
(b) transferring the solution to a container subjected to the ambient pressure and the ambient temperature;
(c) submerging said fatigued muscles or damaged tissue into the solution in the container;
(d) adding energy from an energy source to the solution to invoke nucleation of molecular oxygen containing microbubbles and liberation of the molecular oxygen from the solution in proximity to the fatigued muscles or damaged tissue; and
(e) maintaining the fatigued muscles or damaged tissue in the solution to invigorate the muscle or repair the tissue by action of the oxygen.

47. The method of claim 46 wherein the energy added to the solution comprises heat energy supplied to the solution.

48. The method of claim 46 wherein the energy source for adding energy to the solution is mechanical circulation of the solution.

49. The method of claim 46 wherein the molecular oxygen is air or pure molecular oxygen.

50. The method of claim 46 wherein the step of transferring the solution to a container comprises gradually reducing the pressure of the solution to minimize turbulent conditions and maintain the concentration of dissolved oxygen in solution above 100 mg/l during the transfer.

51. The method of claim 46 further comprising the step of maintaining the ambient pressure between 0.9 atm and 1.1 atm, and the ambient temperature between 65° F. and 72° F.

52. The method of claim 46 wherein the step of dissolving the molecular oxygen into the liquid comprises pumping the liquid through a conduit and injecting the gas into the pumped liquid at supersonic speeds.

53. A method of treating a fatigued muscles comprising the steps of:
(a) dissolving a molecular oxygen into water to provide a hypersaturated solution containing dissolved molecular oxygen substantially resistant to homogenous nucleation of molecular oxygen under static conditions in an ambient pressure and an ambient temperature environment;
(b) transferring the solution to an environment having the ambient pressure and the ambient temperature while adding a minimal amount of energy to the solution so as to maintain the concentration of dissolved gas in the solution as it is transferred to the environment;
(c) immersing the fatigued muscles into the hypersaturated solution;
(d) adding energy from an energy source to the solution to induce nucleation of molecular oxygen microbubbles and liberation of the molecular oxygen from the solution; and
(e) maintaining the muscles in the solution to invigorate them by action of the oxygen.

54. The method of claim 53 wherein the energy added to the solution comprises heat energy supplied to the solution.

55. The method of claim 53 wherein the heat energy added to the solution comprises heat dissipating from the tissue cells.

56. The method of claim 53 wherein the energy source for adding energy to the solution is mechanical circulation of the solution.

57. The method of claim 53 wherein the energy source for adding energy to the solution originates from surface forces.

58. The method of claim 53 wherein the molecular oxygen is obtained from air or pure oxygen.

59. The method of claim 53 wherein a solid surface is submerged in the solution to stimulate heterogeneous nucleation of the molecular oxygen containing bubbles.

60. The method of claim 53 wherein the concentration of dissolved molecular oxygen in solution as it is transferred into the container is above 100 mg/l.

61. The method of claim 53 wherein, in the dissolving step, the solution of dissolved molecular oxygen is hypersaturated and has a dissolved molecular oxygen concentration above 100 mg/l.

62. The method of claim 58 comprising the step of maintaining the ambient pressure between 0.9 atm and 1.1 atm, and the ambient temperature between 65° F. and 72° F.
